(19) United States
(12) Patent Application Publication
(10) Pub. No.: US 2024/0009157 A1
(43) Pub. Date: Jan. 11, 2024

(54) 3,5-DIIODOTHYROPROPIONIC ACID COMPOSITIONS AND METHODS OF USE THEREOF

(71) Applicant: PriZm, LLC, Chandler, AZ (US)

(72) Inventors: Vivek KOPPARTHI, Chandler, AZ (US); Kiran AVANCHA, Chandler, AZ (US)

(21) Appl. No.: 18/350,099

(22) Filed: Jul. 11, 2023

Related U.S. Application Data
(60) Provisional application No. 63/388,241, filed on Jul. 11, 2022.

(51) Int. Cl. A61K 31/192; A61P 25/28; A61K 9/00; A61K 9/20

(52) U.S. Cl. CPC A61K 31/192; A61P 25/28; A61K 9/0095; A61K 9/2054; A61K 9/2013; A61K 9/2009; A61K 9/2072

(57) ABSTRACT

The present subject matter is directed to pharmaceutical compositions comprising 3,5-diiodothyropropionic acid, or a salt thereof, and one or more pharmaceutically acceptable excipients. The present subject matter is further directed to methods of treating Allan-Herndon-Dudley syndrome comprising administering to a subject in need thereof one or more compositions comprising 3,5-diiodothyropropionic acid, or salt thereof, and one or more pharmaceutically acceptable excipients.

### CROSS-REFERENCE TO RELATED APPLICATION

This application claims the benefit of U.S. Provisional Patent Application No. 63/388,241, filed on Jul. 11, 2022, the content of which is incorporated herein by reference in its entirety.

### TECHNICAL FIELD

The present subject matter is directed to pharmaceutical compositions comprising 3,5-diiodothyropropionic acid, or a salt thereof, and one or more pharmaceutically acceptable excipients. The present subject matter is further directed to methods of treating Allan-Herndon-Dudley syndrome comprising administering compositions comprising 3,5-diiodothyropropionic acid, or salt thereof, and one or more pharmaceutically acceptable excipients to a subject in need thereof.

### BACKGROUND ART

Allan-Herndon-Dudley Syndrome (“AHDS”) is an X-linked recessive developmental disorder causing intellectual disability and movement issues in males. Specifically, patients with AHDS have a mutant SLC16A2 gene resulting in a malformed monocarboxylate transporter 8 (“MCT8”) protein. Symptoms of AHDS are caused by a lack of cellular uptake of the thyroid hormone triiodothyronine (“T3”), which is normally transported across the cell membrane by MCT8. This MCT8 deficiency leads to a lack of T3 in tissues that need T3 to function properly contributing to an accumulation of T3 in the blood serum. The other thyroid hormone thyroxine (“T4”) usually remains at normal serum levels in AHDS patients but may also be slightly reduced from a normal level. Thyroid stimulating hormone (“TSH”) is normal to slightly elevated in AHDS patients.

Currently, no treatment for AHDS has been approved by the United States Food and Drug Administration. Clinical trials have been completed for the drug, triiodothyroacetic acid (“TRIAC”), for use in the treatment of AHDS. However, TRIAC shares a close structural similarity to T3, which makes it difficult to accurately assess T3 serum levels. Further, TRIAC has been shown to significantly reduce T4 serum levels.

3,5-diiodothyropropionic acid (“DITPA”) is another thyroid hormone analog that has been studied for treatment of AHDS. However, as mentioned above, DITPA has not been approved for use in the treatment of AHDS. This lack of approval may be due to a lack of effective dosing regimens, stable and effective compositions and extensive pharmacological assessments. While WO/2012/171065, published Dec. 20, 2012, attempts to establish DITPA dosing regimens for AHDS patients, this publication offers only theoretical examples.

Thus, there is a need in the art for stable and effective compositions containing DITPA.

### DISCLOSURE

The present subject matter is directed to pharmaceutical compositions comprising 3,5-diiodo-thyropropionic acid (“DITPA”) and one or more pharmaceutically acceptable excipients.

The present subject matter is further directed to methods of treating Allan-Herndon-Dudley syndrome (“AHDS”) or one or more symptoms of AHDS comprising administering compositions comprising DITPA and one or more pharmaceutically acceptable excipients to a subject in need thereof.

### BRIEF DESCRIPTION OF THE DRAWINGS

FIG. 1 is a graph showing the dose response for DITPA administration to liver in vitro, and its effect on D1 enzymatic activity, i.e., converting T4 to T3.

FIG. 2 is a picture of quadrisected scored tablets.

FIG. 3 is a schematic depicting a scored tablet, indicating application of force to break the tablet in half, or to break the tablet half into quarter portions of the whole tablet.

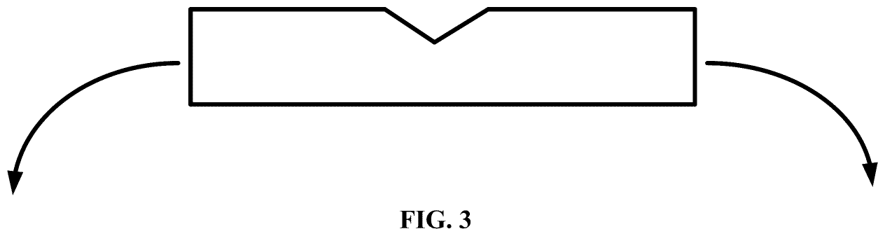

### DESCRIPTION OF EMBODIMENTS

The Applicant has discovered compositions of 3,5-diiodothyropropionic acid (“DITPA”) that are surprisingly stable, and surprisingly effective for the treatment of Allan-Herndon-Dudley Syndrome (“AHDS”).

In one embodiment, the present subject matter is directed to pharmaceutical compositions comprising DITPA, or a salt thereof, and one or more pharmaceutically acceptable excipients.

In a preferred embodiment, DITPA, or a salt thereof, may present in the pharmaceutical compositions of the present subject matter at a concentration from about 0.001% to about 10% w/w or w/v.

In a preferred embodiment, the one or more pharmaceutically acceptable excipients may be present in the pharmaceutical compositions of the present subject matter at a concentration from about 90% to about 99.999% w/w or w/v.

Pharmaceutically acceptable excipients suitable for use in the present formulations include, but are not limited to, disintegrants, binders, fillers, plasticizers, lubricants, permeation enhancers, surfactants, sweeteners, sweetness enhancers, flavoring agents and pH adjusting agents.

The term “disintegrants” as used herein refers to pharmaceutically acceptable excipients that facilitate the disintegration of the tablet once the tablet contacts water or other liquids.

Disintegrants suitable for use in the present formulations include, but are not limited to, natural starches, such as maize starch, potato starch etc., directly compressible starches such as starch 1500, modified starches such as carboxymethyl starches, sodium hydroxymethyl starches and sodium starch glycolate and starch derivatives such as amylose, cross-linked polyvinylpyrrolidones such as crospovidones, modified celluloses such as cross-linked sodium carboxymethyl celluloses, sodium hydroxymethyl cellulose, calcium hydroxymethyl cellulose, low-substituted hydroxypropyl cellulose, alginic acid, sodium alginate, microcrystalline cellulose, methacrylic acid-divinylbenzene copolymer salts and combinations thereof.

Binders suitable for use in the present formulations include, but are not limited to, polyethylene glycols, soluble hydroxyalkyl celluloses, polyvinylpyrrolidone, gelatins, natural gums and combinations thereof.

Fillers suitable for use in the present formulations include, but are not limited to, dibasic calcium phosphate, calcium phosphate tribasic, calcium sulfate and dicalcium sulfate, lactose, sucrose, amylose, dextrose, mannitol, inositol and combinations thereof.

Plasticizers suitable for use in the present formulations include, but are not limited to, microcrystalline cellulose, triethyl citrate, poly-hexanediol, acetylated monoglyceride, glyceryl triacetate, castor oil, and combinations thereof.

Lubricants suitable for use in the present formulations include, but are not limited to, magnesium stearate, sodium stearyl fumarate, stearic acid, glyceryl behenate, micronized polyoxyethylene glycol, talc, and combinations thereof.

Permeation enhancers suitable for use in the present formulations include, but are not limited to, precipitated silicas, maltodextrins, P-cyclodextrins menthol, limonene, carvone, methyl chitosan, polysorbates, sodium lauryl sulfate, glyceryl oleate, caproic acid, enanthic acid, pelargonic acid, capric acid, undecylenic acid, lauric acid, myristic acid, palmitic acid, oleic acid, stearic acid, linolenic acid, arachidonic acid, benzethonium chloride, benzethonium bromide, benzalkonium chloride, cetylpyridium chloride, edetate disodium dihydrate, sodium desoxycholate, sodium deoxyglycolate, sodium glycocholate, sodium caprate, sodium taurocholate, sodium hydroxybenzoyal amino caprylate, dodecyl dimethyl aminopropionate, L-lysine, glycerol oleate, glyceryl monostearate, citric acid, peppermint oil and combinations thereof. Surfactants suitable for use in the present formulations include, but are not limited to, sorbitan esters, docusate sodium, sodium lauryl sulphate, cetriride and combinations thereof.

Sweeteners suitable for use in the present formulations include, but are not limited to, aspartame, saccharine, potassium acesulfame, sodium saccharinate, neohesperidin dihydrochalcone, sucralose, sucrose, dextrose, mannitol, glycerin, xylitol, and combinations thereof.

Sweetness enhancers suitable for use in the present formulations include, but are not limited to, ammonium salt forms of crude and refined glycyrrhizic acid.

Flavoring agents suitable for use in the present formulations include, but are not limited to, peppermint oil, menthol, spearmint oil, citrus oil, cinnamon oil, strawberry flavor, cherry flavor, raspberry flavor, orange oil and combinations thereof.

pH adjusting agents suitable for use in the present formulations include, but are not limited to, hydrochloric acid, citric acid, fumaric acid, lactic acid, sodium hydroxide, sodium citrate, sodium bicarbonate, sodium carbonate, ammonium carbonate, sodium acetate and combinations thereof. In another preferred embodiment, the pharmaceutical compositions of the present formulations do not contain a preservative.

Pharmaceutical compositions of the present formulations may be formulated in any dosage form including but not limited to aerosol including metered, powder and spray, chewable bar, bead, capsule including coated, film coated, gel coated, liquid filled and coated pellets, cellular sheet, chewable gel, concentrate, elixir, emulsion, film including soluble, film for solution and film for suspension, gel including metered gel, globule, granule including granule for solution, granule for suspension, chewing gum, inhalant, injectable including foam, liposomal, emulsion, lipid complex, powder, lyophilized powder and liposomal suspension, liquid, lozenge, ointment, patch, electrically controlled patch, pellet, implantable pellet, pill, powder, powder, metered powder, solution, metered solution, solution concentrate, gel forming solution/solution drops, spray, metered spray, suspension, suspension, syrup, tablet, chewable tablet, coated tablet, coated particles in a tablet, film coated tablet, tablet for solution, tablet for suspension, orally disintegrating tablet, soluble tablet, sugar coated tablet, dispersible tablet, tablet with sensor, tape, troche and wafer and extended release and delayed release forms thereof.

In a preferred embodiment, the pharmaceutical compositions of the present formulations are in tablet form. In a more preferred embodiment, the pharmaceutical compositions of the present formulations are in a dispersible tablet form. In an even more preferred embodiment, the pharmaceutical compositions of the present formulations are in a water-dispersible tablet form. In a most preferred embodiment, the pharmaceutical compositions of the present formulations are in a water-dispersible tablet form wherein the tablet is scored such that the tablet is dividable into four equal parts.

In a preferred embodiment, when the pharmaceutical compositions of the present formulations are in a water-dispersible tablet form the tablet dispersion time is about 70 seconds or less, more preferably about 60 seconds or less and even more preferably about 40 seconds or less, when the tablet is placed in at least one teaspoon of water.

In another embodiment, the present subject matter is directed to a method of treating Allan-Herndon-Dudley syndrome (“AHDS”) comprising administering compositions comprising DITPA, or a salt thereof, and one or more pharmaceutically acceptable excipients to a subject in need thereof. In another embodiment, the present subject matter is directed to a method of treating one or more symptoms of AHDS comprising administering compositions comprising DITPA, or a salt thereof and one or more pharmaceutically acceptable excipients to a subject in need thereof.

In a preferred embodiment, the compositions of the present subject matter are administered to a subject in need thereof once a day, more preferably twice a day and most preferably three times a day. Each administration may be one or more full tablets, or a portion of a tablet, such as ½ or ¼ of a whole tablet.

In another preferred embodiment, the compositions of the present subject matter are administered at a dosage of from about 0.1 to about 10 milligrams per kilogram of body weight of the subject per day (“mg/kg/day”), more preferably from about 1 to about 5 mg/kg/day and most preferably at about 2.5 mg/kg/day.

In a preferred embodiment, DITPA is administered to a subject that is less than 18 years old. In another preferred embodiment, DITPA is administered to a pregnant mother of a subject in need thereof.

In a preferred embodiment, the pharmaceutical compositions of the present subject matter are administered orally to the subject.

As used herein the term “pharmaceutically acceptable” refers to ingredients that are not biologically or otherwise undesirable in an oral application.

As used herein, all numerical values relating to amounts, weights, and the like, are defined as “about” each particular value, that is, plus or minus 10%. For example, the phrase “10% w/w” is to be understood as “9% to 11% w/w.” Therefore, amounts within 10% of the claimed value are encompassed by the scope of the claims.

As used herein “% w/w” refers to the weight percent by weight of the total formulation. As used herein “% w/v” refers to the weight percent by volume of the total formulation. As used herein the term “effective amount” refers to the amount necessary to treat a subject in need thereof.

As used herein the term “treatment” or “treating” refers to alleviating or ameliorating AHDS or symptoms of AHDS.

As used herein, the term “stable” includes, but is not limited to, physical and chemical stability. Pharmaceutically acceptable salts of that can be used in accordance with the current subject matter include but are not limited to hydrochloride, dihydrate hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, mesylate, maleate, gentisinate, fumarate, tannate, sulphate, tosylate, esylate, gluconate, glucuronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts.

Throughout the application, the singular forms “a,” “an,” and “the” include plural reference unless the context clearly dictates otherwise.

The disclosed embodiments are simply exemplary embodiments of the inventive concepts disclosed herein and should not be considered as limiting unless the claims expressly state otherwise.

The following examples are intended to illustrate the present subject matter and to teach one of ordinary skill in the art how to use the formulations of the subject matter. They are not intended to be limiting in any way.

### EXAMPLES

### Example 1—Sample Formulation (Prophetic)

TABLE 1
Dry Powder Drug Product Components and Composition

| | | | Composition | |
|---|---|---|---|---|
| | Quality | | (mg/Tablet) | |
| Component | Standard | Function | 4 mg | 10 mg |
| SRW101 | In-house | Active | 4 | 10 |

| | | | |
|---|---|---|---|
| Microcrystalline Cellulose | USP | Binder | 10-25% |
| | USP | Bulking Agent | 10-20% |
| Magnesium Stearate | USP/NF | Lubricant | 0.5-1.0% |
| Croscarmellose | USP | Disintegrant | 5-10% |
| Citric Acid | USP | pH modifier/ | 1-2% |
| | | preservative | |
| Silicon Dioxide | USP | Glidant | 2-5% |
| Flavoring Agent | IIG listed | Taste masking | QS |

IIG: FDA's Inactive Ingredients database;
NF: National Formulary;
USP: US pharmacopeia

### Example 2—Administration of a Water-dispersible Tablet (Prophetic)

### Method

A water-dispersible tablet of the present subject matter, such as, for example, a tablet as in Example 1 (Table 1) above, typically completely dissolves in water, with mixing, in about 60 seconds or less. The resulting dispersion is then immediately orally administered to an infant.

### Results

The dispersion is imbibed by the infant and generally is well tolerated.

### Example 3—Dosing Regimen for a Pediatric Subject (Prophetic)

### Method

3,5-diiodothyropropionic acid (“DITPA”) was administered to a pediatric patient suffering from Allan-Herndon-Dudley Syndrome at a daily dosage of 1 mg/kg/day divided over three administration spaced 8 hours apart for 2 weeks. Following the first 2 weeks, the daily dosage was increased to 2 mg/kg/day for 2 additional weeks. Following the 2 additional weeks, T3 serum levels were assessed. The patient was found to have T3 serum levels more than 15% below normal. The patient was then administered DITPA at a daily dosage of 1.5 mg/kg/day for 28 days at which time T3 serum levels were reassessed. Upon reassessment T3 serum levels were normal.

### Results

The dosing regimen allows successfully identified proper dosing for the pediatric patient to maintain normal T3 serum levels.

### Example 4—Dosing Regimen for a Pediatric Subject (Prophetic)

### Method

DITPA was administered to a pediatric patient suffering from Allan-Herndon-Dudley Syndrome at a daily dosage of 1 mg/kg/day divided over three administration spaced 8 hours apart for 2 weeks. Following the first 2 weeks, the daily dosage was increased to 2 mg/kg/day for 2 additional weeks. Following the 2 additional weeks, T3 serum levels were assessed. The patient was found to have T3 serum levels more than 15% above normal. The patient was then administered DITPA at a daily dosage of 2.5 mg/kg/day for 28 days at which time T3 serum levels were reassessed. Upon reassessment T3 serum levels were normal.

### Results

The dosing regimen allows successfully identified proper dosing for the pediatric patient to maintain normal T3 serum levels.

### Example 5—Dosing Regimen for a Pediatric Subject (Prophetic)

### Method

DITPA was administered to a pediatric patient suffering from Allan-Herndon-Dudley Syndrome at a daily dosage of 1 mg/kg/day divided over three administration spaced 8 hours apart for 2 weeks. Following the first 2 weeks, the daily dosage was increased to 2 mg/kg/day for 2 additional weeks. Following the 2 additional weeks, T3 serum levels were assessed. The patient was found to have T3 serum levels more than 15% below normal. The patient was then administered DITPA at a daily dosage of 1.5 mg/kg/day for 28 days at which time T3 serum levels were reassessed. Upon reassessment T3 serum levels were again found to be more than 15% below normal. The patient was then administered DITPA at a daily dosage of 1.0 mg/kg/day for 28 days at which time T3 serum levels were reassessed. Upon reassessment T3 serum levels were found to be normal.

### Results

The dosing regimen allows successfully identified proper dosing for the pediatric patient to maintain normal T3 serum levels.

### Effect of DITPA on Removal of G-Tube

One child who started DITPA while having a G-tube, gained weight and the g-tube was removed. We designed a proposed Phase 3 study to be robust with endpoints intended to determine the clinical benefit of DITPA versus surrogate endpoints.

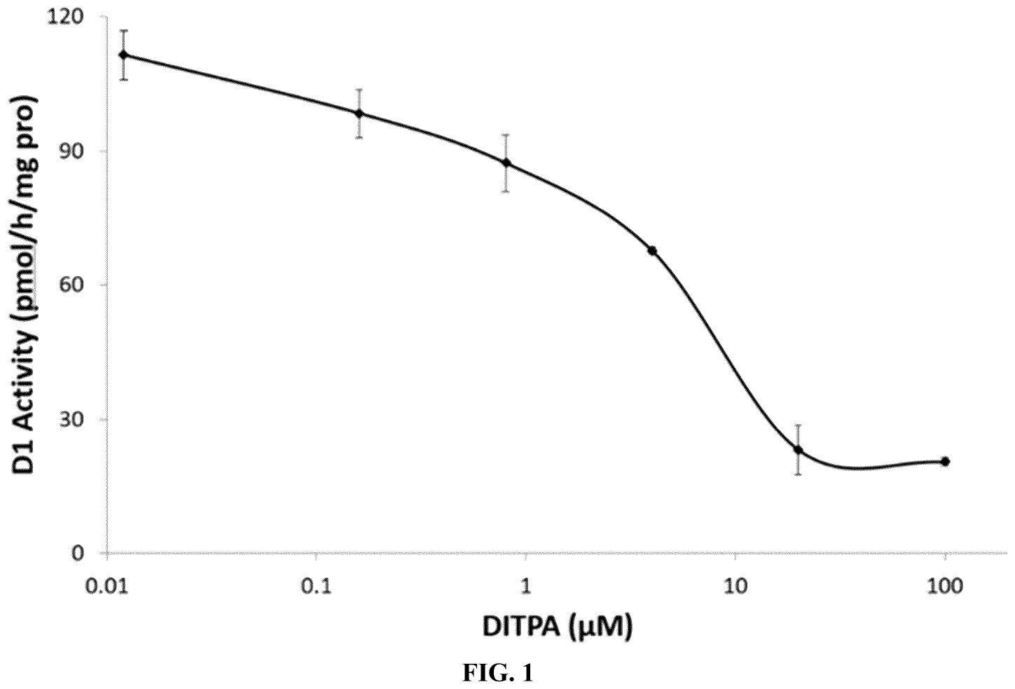

Below are the specific endpoints of the planned Phase 3 study and associated rationales:

| FDA | |
|---|---|
| Concurred | |
| Endpoints | Expected Outcome with Justification |
| Neurologic | Studies assessing the neurological and behavioral deficiencies |
| Developmental | in MCT8-mutant (mct82/2) showed that DITPA (SRW101) and other |
| (Age | TH analogs restored the myelin and axon outgrowth deficiencies in |
| Appropriate) | mct82/2 larvae (Zada et al 2014). These studies also showed that the |
| CHOP- | SRW101 and other TH analogs partially rescued the hypomyelination |
| INTEND total | in the CNS of MCT8 mutant (mct8−/−) zebrafish (Zada et al 2016). |
| score | These studies in particular show that administration of |
| Head | SRW101 (and other TH analogs) early in infant development can |
| Control Scale | specifically reduce neurologic damage in patients with AHDS. It is |
| total score | noted that the TH analog Triac has also been studied in pediatric |
| Gross Motor | subjects with positive effects. |
| Function | Zada D, Tovin A, Lerer-Goldshtein T, Vatine G D, and |
| Measure | Appelbaum L (2014). Altered Behavioral Performance and Live |
| (GMFM)-88 | Imaging of Circuit-Specific Neural Deficiencies in a Zebrafish Model |
| | for Psychomotor Retardation. PLoS Genet. 10(9): e1004615. |
| | Zada D, Tovin A, Lerer-Goldshtein T, Vatine G D, and |
| | Appelbaum L (2016). Pharmacological treatment and BBB-targeted |
| | genetic therapy for MCT8-dependent hypomyelination in zebrafish. |
| | Disease Models & Mechanisms. 9, 1339-1348. |
| Endocrine and | Studies assessing the metabolic effects of MCT8 deficiency |
| Metabolism | using MCT8-deficient (knockout) mice (Mct8KO). These studies |
| (I) | showed that SRW101 normalized all measurements and other |
| | parameters of TH action, and that SRW101 is relatively MCT8 |
| | independent for entry into the brain and corrects the TH deficit in |
| | Mct8KO mice without causing thyrotoxic effect in the liver (Di |
| | Cosmo et al 2009); and analysis of TH target genes revealed |
| | amelioration of the thyrotoxic state in the liver (ameliorating |
| | hypermetabolism). |
| | Ferrara A M, Liao X, Ye H, Weiss R E, Dumitrescu |
| | A M, and Refetoff S (2015). The thyroid hormone analog DITPA |
| | ameliorates metabolic parameters of male mice with Mct8 deficiency. |
| | Endocrinology. 156: 3889-3894. |
| | Di Cosmo C, Liao X H, Dumitrescu A M, Weiss R E, |
| | and Refetoff S (2009). A thyroid hormone analog with reduced |
| | dependence on the monocarboxylate transporter 8 for tissue transport. |
| | Endo. 150(9): 4450-4458. |
| Endocrine and | These are the consequences of the normalization of serum T3 |
| Metabolism | levels (first primary endpoint) as they measure the anticipated |
| (II) | metabolic changes resulting from the normalization of the thyroid |
| | tests. More specifically, the reduction of T3, which acts on peripheral |
| | tissue to accelerate the metabolism, is expected to improve nutrition |
| | and increase the ability to gain weight. |
| | Important measurements such as weight gain (corrected for |
| | age) and metabolic parameters (cholesterol, creatine kinase, SHBG) |
| | are secondary endpoints. |
| | Annotated observations by the parents such as sleep, food |
| | record, motor activity, are of immense value. |
| | See FIG. 1. Dose response of DITPA added to liver in vitro |
| | and measurement of D1 enzymatic activity (conversion of T4 to T3) |
| | in vitro evidence of direct effect of DITPA in decreasing the T3 |
| | generated from T4, rather than reducing it through decrease in T4 by |
| | TSH suppression, as is the case with TRIAC. T4 is important to the |
| | brain even in the presence of reduced uptake due to MCT8 deficiency. |
| Measure of | MCT8-deficient mice have increased energy expenditure and |
| decreasing | reduced fat mass that is abrogated by normalization of serum T3 |
| thyrotoxicosis | levels (Di Cosmo et al 2013). Clinically, children with MCT8 |
| Improvement | deficiency lose weight, even when adequately nourished. Changes in |
| in in body | serum markers of thyroid hormone (TH) action compatible with |
| weight, basal | thyrotoxicosis suggested that this might be due to T3 excess in |
| metabolic | peripheral tissues. We used MCT8-deficient mice as they replicate the |
| index (BMI) | human thyroid phenotype and are thus suitable for metabolic studies |
| Avoidance of | that were unavailable in humans. |
| feeding tube | As compared to wild-type mice, MCT8KO mice were leaner |
| Change in | due to reduced fat mass. They tended to use more carbohydrates and |
| head | fewer lipids during the dark phase. MCT8KO mice had increased total |
| circumference | energy expenditure (TEE) and food and water intake, with normal |
| and | total activity, indicating hypermetabolism. To determine whether this |
| Decrease in | is due to the high serum T3, we studied mice deficient in both MCT8 |
| frequency of | and deiodinase 1 (Mct8D1KO) with serum T3 similar to wild type |
| dyskinetic | mice and wild type mice given L-T3 to raise their serum T3 to the |
| episodes. | level of Mct8KO mice. Contrary to MCT8KO, MCT8D1KO mice had |
| | similar fat mass, TEE, and food intake as their DIKO littermates, |
| | whereas T3-treated wild type mice showed increased food intake and |
| | TEE, similar to MCT8KO mice. In skeletal muscle, MCT8KO mice |
| | had increased T3 content and TH action and increased glucose |
| | metabolism, which improved in MCT8D1KO mice. These studies |
| | indicate that the high serum T3 in MCT8 deficiency increases the |
| | TEE and fails to maintain weight despite adequate calorie intake. This |
| | is mediated by tissues that are not predominantly MCT8 dependent |
| | for TH transport, including skeletal muscle. Normalizing serum T3 |
| | level by deleting deiodinase 1 corrects body composition and the |
| | metabolic alterations caused by the MCT8 deficiency (Di Cosmo et al |
| | 2013). |
| | Di Cosmo C, Liao X H, Ye H, Ferrara A M, Weiss R E, |
| | Refetoff S, and Dumitrescu A M (2013). Mct8-deficient mice have |
| | increased energy expenditure and reduced fat mass that is abrogated |
| | by normalization of serum T3 levels. Endocrinology. 154, 4885-4895. |

Our primary endpoint was chosen to ensure high probability of NDA success based on following factors:
Our estimated PTRS for reaching primary endpoint based on T3 level difference at the end of randomized withdrawal period has >99% power to detect a change of at least 100 ng/dL in serum T3 levels from baseline (start of randomized withdrawal) to week 8 (week 34 of trial) vs. placebo. We know from prior studies (such as LT3 treatment in primary hyperthyroidism) that LT3 levels increase within hours after treatment and therefore in the 8-week period T3 levels in MCT8 deficient patients off SRW101 treatment should have ample time increase sharply and return to baseline high within days.
The key secondary endpoint is to assess the complete total T3, free T4, and TSH response rate at the end of the dose-titration and maintenance treatment with SRW-101 in the initial single-arm, open label part of the study (Week 24) in the mITT population. The key secondary null hypothesis is that the proportion of patients who are total T3, free T4, and TSH complete responders at the Week 24 Visit is less than or equal to 0.2 The alternative hypothesis is that the proportion of patients who are total T3, free T4, and TSH complete responders at the Week 24 Visit is greater than 0.2. The exact test for one proportion will be used. Efficacy of SRW-101 will be declared when the proportions of responders at the Week 24 Visit is statistically significantly greater than 0.2 at a one-sided alpha level of 0.025. A sample size of 40 patients age 0-17 years will have nearly 100% power to detect a difference of 100 ng/dL using a one-sided exact test for one proportion with a target significance level of 0.025. For the secondary outcomes, it is assumed that the population proportion under the null hypothesis is 0.2 and the alternative hypothesis is 0.80.
The number and proportions (expressed as percentages) of total T3, free T4, and TSH responders at each scheduled time point during the OLDT period and OLDM period, including the Week 24 Visit will be calculated. These proportions, along with their exact (Clopper-Pearson) 95% CIs, will be summarized by scheduled time point. Enrolled patients who had missing thyroid function test assessment at Week 24 will be counted as non-responders for the key secondary endpoint. Other secondary endpoint analyses will be specified in the statistical analysis plan (SAP) and approximate powers will be calculated then.

### Example 6—Dosage Form, Route of Administration, and Dosing Regimen

The drug product is a tablet for suspension (i.e., dispersible tablet) designed to rapidly disintegrate in a small amount of aqueous media prior to administration. Each tablet strength may be quadrisectally scored to allow for administration of dosing of half or quarter tablets. This allows, for example with a 4 mg tablet, doses as low as 1 mg increments. A summary of the exemplary drug product attributes is presented in Table 2.

TABLE 2
Exemplary Drug Product Attributes

| Dosage form | Tablet for suspension |
|---|---|
| Strengths | 4 and 10 mg quadrisected scored tablets allowing for associated dosing: |
| | 4 mg: scored for 1, 2, and 4 mg portions |
| | 10 mg: scored for 2.5, 5, and 10 mg portions |
| Process/route of | Oral, after tablet or tablet portion is disintegrated and suspended in |
| Administration | liquid |
| Dosing regimen | The starting dose is 2.0 mg/kg/day TID with dose titration based on |
| | serum T3 levels |

T3: triiodothyronine; TID: ter die (three times a day)

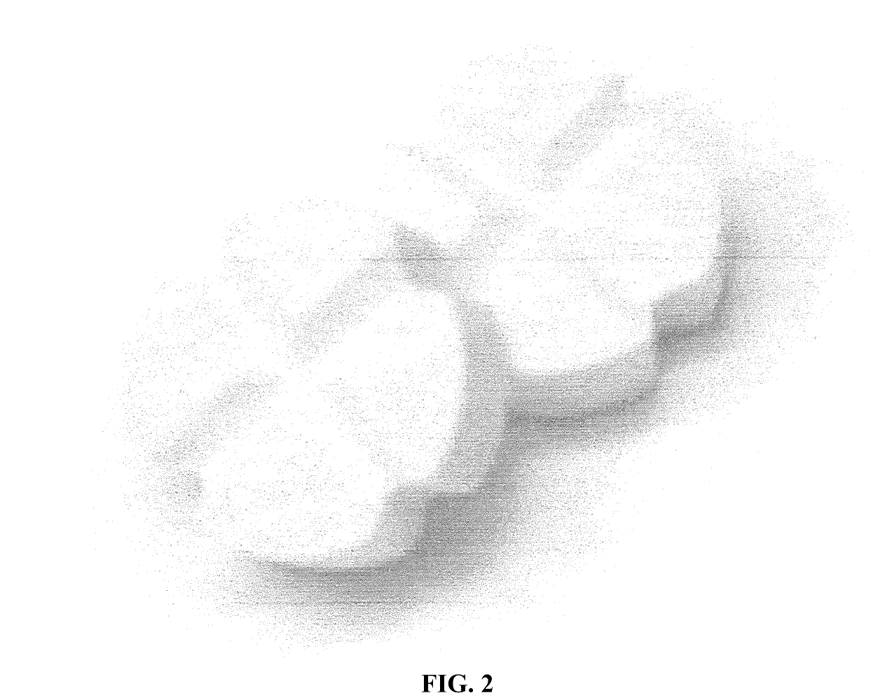

One intended commercial product will be a tablet for suspension (dispersible tablet) that is quadrisectally scored to allow partitioning of the tablet into halves or fourths, to allow a 4 mg tablet to be broken easily into 1 mg increments for dosing. FIG. 2 is a picture of an example of quadrisectally scored tablets.

Two product strengths (4 and 10 mg) could be developed that will allow flexible dosing across the anticipated dose ranges for study PRZ-MCT8-101 and for commercial use. These two tablets, quadrisectally scored, provide easy dose portions of 1 mg, 2 mg, 2.5 mg, 4 mg, 5 mg, and 10 mg. Of course, multiple portions can be combined to provide additional dosage amounts, such as combining a 1 mg portion with a 2 mg portion to provide a 3 mg dose, or combining a 5 mg portion with a 2 mg portion to provide a 7 mg dose.

Qualification of the scored tablet will be characterized in accordance with FDA Guidance for Industry: Tablet Scoring: Nomenclature, Labeling, and Data for Evaluation (March 2013) and the specific studies outlined in EOP2/Pre-phase 3 FDA meeting package.

### Drug Product and Administration

### Components and Composition

The drug product may be a tablet for suspension (dispersible tablet) quadrisectally scored so that it can be split into halves or fourths. See, for example, FIG. 2. The tablet, or portion of tablet if halved or quartered, is dispersed in a small amount of aqueous media (e.g., 5-10 mL water) prior to oral administration of the suspension. Two different product strengths are planned (i.e., 4 and 10 mg) which will be appropriately differentiated by color debossment.

The drug product components and composition are summarized in Table 3 below. All excipients are well-established for oral products and are at levels well below the respective maximum potencies listed in FDA's Inactive Ingredients (IIG) database. Therefore, there are no novel excipients involved in the manufacture of the drug product.

The primary container closure system will be blister packaging. Based on drug substance and drug product (i.e., capsules) from Titan studies, the drug is stable at ambient temperature and light. While the drug does not appear to have hygroscopicity, the formulation itself will be evaluated accordingly to assess for any need for moisture mitigation.

TABLE 3
SRW101 Dry Powder Drug Product Components and Composition

| | | | Composition | |
|---|---|---|---|---|
| | Quality | | (mg/Tablet) | |
| Component | Standard | Function | 4 mg | 10 mg |
| SRW101 | In-house | Active | 4 | 10 |

| | | | |
|---|---|---|---|
| Microcrystalline | USP | Binder | 10-25% |
| Cellulose | | | |
| | USP | Bulking Agent | 10-20% |
| Magnesium Stearate | USP/NF | Lubricant | 0.5-1.0% |
| Croscarmellose | USP | Disintegrant | 5-10% |
| Citric Acid | USP | pH modifier/ | 1-2% |
| | | preservative | |
| Silicon Dioxide | USP | Glidant | 2-5% |
| Flavoring Agent | IIG listed | Taste masking | QS |

IIG: FDA's Inactive Ingredients database;
NF: National Formulary;
USP: US pharmacopeia

### Example 7—The Manufacturing Process

One exemplary manufacturing process consists of compounding and blending SRW101 and excipients stepwise in a tote bin blender to form a common 10 kg blend (commercial batch size). From the common blend, the 4 and 10 mg tablets may be compressed using, for example, an IMA Comprima tablet press.

Exemplary lots will be manufactured at full scale (10 kg) using the intended commercial manufacturing process. The 4 and 10 mg product strengths will be manufactured from the same 10 kg blend.

### Specification and Analytical Methods

The exemplary drug product specification is provided in Table 4. The specification will be developed in accordance with ICH-Q6A. All non-compendial methods (i.e., in-house HPLC for assay, degradation products, and dissolution sample testing) would be fully validated prior to the release of the clinical drug product lots. All compendial methods will be qualified prior to the release of clinical drug product lots. The degradation product method will be validated to be stability indicating with forced degradation studies (i.e., heat, light, acid, base, and oxidation).

The limits for degradation products will be qualified for safety in accordance with ICH-Q3B Impurities in New Drug Products.

TABLE 4
SRW101 Dry Powder Drug Product Specification

| Test | Method | Limits |
|---|---|---|
| Description | Visual | TBD |
| Identification | | |
| TBD | TBD | TBD |
| HPLC | In-house | Retention time of the major peak |
| | method | in the chromatogram of the assay. |
| | | Preparation corresponds to that in |
| | | the chromatogram of the standard. |
| | | Preparation obtained as specified |
| | | in the assay. |
| Content uniformity | USP<905> | Meets USP requirements |
| Water content | USP <921>, | TBD |
| | Method lc | |
| Assay | In-house | Release: 95.0-105.0% LC |
| | method | Shelf-life: 90.0-110.0% LC |
| Dissolution | USP <711> | Q: NLT 85% at 15 minutes |
| | In-house | |
| | assay | |
| Disintegration | USP <701> | Report results |
| Degradation products | In-house | |
| Specified degradation | method | |
| products | | |
| TBD (if necessary) | | Report results (%) |
| Unspecified | | |
| degradation products | | |
| Any individual | | NMT 0.2% |
| unspecified | | |
| Total | | Report results (%) |
| Microbial limits | USP <61> | |
| Total aerobic | | NMT 1000 cfu/g |
| microbial count | | |
| Total yeasts and | | NMT 100 cfu/g |
| molds count | | |
| Specified organisms | USP <62> | |
| E. coli | | Absence in 10 g |

NLT: Not less than;
NMT: not more than;
TBD: to be determined;
USP: US Pharmacopeia

### Characterization of Tablet Dispersion and Tablet Splitting

In addition to dispersion of the tablet in water, studies will be performed to evaluate and ensure the adequate dispersion (physical tests) of divisible portions of the tablet in other vehicles (i.e., formula milk and apple sauce).

The tablet is intended to be hand split in halves or quarters by breaking along the scored lines cut into the surface of the tablet. See FIG. 2. The tablet may be held between the thumb and index finger on either side of the tablet or the tablet half, with the scored lines facing upwards. Downward pressure on the outside of the tablet or tablet half, with upward pressure in the middle, will break the tablet along the lines as shown in FIG. 2.

### Stability

The drug product manufactured with the reprocessed drug substance batch and NDA Registration Lot will be placed in stability studies under long-term, intermediate, and accelerated environmental conditions as outlined in Table 19.6 and in accordance with ICH-Q1A(R2). Drug product maintained under intermediate conditions will be tested at every time point versus at time of out-of-specification (OOS) results during testing of drug substance under accelerated stability conditions.

Photostability studies will also be performed in accordance with ICH-Q1B. 6-month stability data will be developed for all three stability conditions (i.e., long-term, intermediate, and accelerated conditions) along with photostability data.

It is to be understood that the DITPA formulations and regimen for treating AHDS or a symptom of AHDS are not limited to the specific embodiments described above but encompass any and all embodiments within the scope of the generic language of the following claims enabled by the embodiments described herein, or otherwise shown in the drawings or described above in terms sufficient to enable one of ordinary skill in the art to make and use the claimed subject matter.

We claim:

1. A pharmaceutical composition comprising 3,5-diiodothyropropionic acid, or a salt thereof, and one or more pharmaceutically acceptable excipients.

2. The composition of claim 1, wherein the 3,5-diiodothyropropionic acid is present at a concentration from about 0.001% to about 10% w/w or w/v, wherein w/w denotes weight by total weight of the composition and wherein w/v denotes weight by total volume of the composition.

3. The composition of claim 1, wherein the one or more pharmaceutically acceptable excipients are selected from the group consisting of disintegrants, binders, fillers, plasticizers, lubricants, permeation enhancers, surfactants, sweeteners, sweetness enhancers, flavoring agents and pH adjusting agents.

4. The composition of claim 3, wherein the disintegrants are selected from the group consisting of natural starches, directly compressible starches modified starches, starch derivatives, cross-linked polyvinylpyrrolidones, modified celluloses, alginic acid, sodium alginate, microcrystalline cellulose, methacrylic acid-divinylbenzene copolymer salts and combinations thereof.

5. The composition of claim 3, wherein the binders are selected from the group consisting of polyethylene glycols, soluble hydroxyalkyl celluloses, polyvinylpyrrolidone, gelatins, natural gums, and combinations thereof.

6. The composition of claim 3, wherein the permeation enhancers are selected from the group consisting of precipitated silicas, maltodextrins, P-cyclodextrins menthol, limonene, carvone, methyl chitosan, polysorbates, sodium lauryl sulfate, glyceryl oleate, caproic acid, enanthic acid, pelargonic acid, capric acid, undecylenic acid, lauric acid, myristic acid, palmitic acid, oleic acid, stearic acid, linolenic acid, arachidonic acid, benzethonium chloride, benzethonium bromide, benzalkonium chloride, cetylpyridium chloride, edetate disodium dihydrate, sodium desoxycholate, sodium deoxyglycolate, sodium glycocholate, sodium caprate, sodium taurocholate, sodium hydroxybenzoyal amino caprylate, dodecyl dimethyl aminopropionate, L-lysine, glycerol oleate, glyceryl monostearate, citric acid, peppermint oil, and combinations thereof.

7. The composition of claim 3, wherein the surfactants are selected from the group consisting of sorbitan esters, docusate sodium, sodium lauryl sulphate, cetriride and combinations thereof.

8. The composition of claim 1, wherein the composition does not contain a preservative.

9. The composition of claim 1, wherein the composition is in tablet form.

10. The composition of claim 9, wherein the tablet is water dispersible.

11. The composition of claim 10, wherein the tablet is scored such that the tablet is dividable into four equal parts.

12. The composition of claim 10, wherein the tablet disperses in water in about 70 seconds or less.

13. The composition of claim 12, wherein the tablet disperses in water in about 40 seconds or less.

14. A method of treating Allan-Herndon-Dudley syndrome, the method comprising administering the composition of claim 1 to a subject in need thereof.

15. A method of treating one or more symptoms of Allan-Herndon-Dudley syndrome, the method comprising administering the composition of claim 1 to a subject in need thereof.

16. The method of claim 14 wherein the composition is administered at a dosage of from about 0.1 to about 10 milligrams per kilogram of body weight of the subject per day (mg/kg/day).

17. The method of claim 15 wherein the composition is administered at a dosage of from about 1 to about 5 mg/kg/day.

18. The method of claim 14 wherein the composition is administered at a dosage of about 2.5 mg/kg/day.

19. The method of claim 15 wherein an amount of the composition to be administered in one day is split into three parts, with one part administered to the subject each of three times a day.
